(19) United States
(12) Patent Application Publication
(10) Pub. No.: US 2013/0274564 A1
(43) Pub. Date: Oct. 17, 2013

(54) APPARATUS AND METHOD FOR PREDICTING UPCOMING STAGE OF CAROTID STENOSIS

(71) Applicants: SAMSUNG ELECTRONICS CO., LTD., Suwon-si (KR); SAMSUNG LIFE WELFARE FOUNDATION, Seoul (KR)

(72) Inventors: Ha-Young Kim, Hwaseong-si (KR); Hye-Jin Kam, Seongnam-si (KR); Sang-Hyun Yoo, Seoul (KR); Ji-Hyun Lee, Hwaseong-si (KR); Yoonho Choi, Seoul (KR); Mira Kang, Seoul (KR); Jeongeuy Park, Seoul (KR); Jidong Sung, Seoul (KR); Heeyoung Shin, Seoul (KR); Sungwon Cho, Seoul (KR); Soojin Cho, Seoul (KR)

(21) Appl. No.: 13/837,912

(22) Filed: Mar. 15, 2013

(30) Foreign Application Priority Data
Mar. 15, 2012 (KR) 10-2012-0026813

(51) Int. Cl. A61B 5/00; A61B 5/02; A61B 5/145; A61B 8/08; A61B 5/0205

(52) U.S. Cl. CPC A61B 5/7264; A61B 8/0891; A61B 8/5223; A61B 5/0205; A61B 5/14546; A61B 5/02007; A61B 5/7275
USPC 600/301; 600/437; 600/481

(57) ABSTRACT

An apparatus and a method predict an upcoming stage of carotid stenosis. The apparatus includes a receiving unit, a cluster determining unit, a risk factor score extracting unit, a prediction model storage unit, and a predicting unit. The method includes receiving a patient's medical test data relating to carotid stenosis; determining a cluster to which the patient's medical test data belong based on a gender of the patient; extracting from the patient's medical test data a risk factor score comprising a result of carotid stenosis ultrasonography; storing a plurality of prediction models used to predict an upcoming stage of carotid stenosis; and obtaining an outcome by applying a value indicating a stage of carotid stenosis corresponding to the result of carotid stenosis ultrasonography and the extracted risk factor score to the prediction model corresponding to the determined cluster among the plurality of prediction models.

### CROSS-REFERENCE TO RELATED APPLICATIONS

This application claims the benefit under 35 USC 119(a) of Korean Patent Application No. 10-2012-0026813 filed on Mar. 15, 2012, the entire disclosure of which is incorporated herein by reference for all purposes.

### BACKGROUND

1. Field

The following description relates to technology for predicting an upcoming stage of carotid stenosis progression at a specific point in time.

2. Description of Related Art

Strokes are divided into ischemic strokes and hemorrhagic strokes. About 20% to 30% of ischemic strokes are attributed to carotid stenosis. An ischemic stroke occurs when an artery to the brain is blocked. The brain depends on its arteries to bring fresh blood from the heart and lungs. The blood carries oxygen and nutrients to the brain, and takes away carbon dioxide and cellular waste. If an artery is blocked, the brain cells (neurons) cannot make enough energy and will eventually stop working. If the artery remains blocked for more than a few minutes, the brain cells may die. This condition is referred to as an ischemic stroke.

Reportedly, various risk factors including age, blood pressure, smoking, cholesterol, diabetes, and obesity affect carotid stenosis. However, existing studies simply analyze statistical differences between a patient group and a healthy group with respect to each risk factor.

In addition, research has been conducted on a method of predicting the occurrence of a stroke as a result of carotid stenosis, but such research has not explored a method of predicting an upcoming stage of carotid stenosis itself.

### SUMMARY

In one general aspect, an apparatus for predicting an upcoming stage of carotid stenosis includes a receiving unit configured to receive a patient's medical test data relating to carotid stenosis; a cluster determining unit configured to determine a cluster to which the patient's medical test data belong based on a gender of the patient; a risk factor score extracting unit configured to extract from the patient's medical test data a risk factor score including a result of carotid stenosis ultrasonography; a prediction model storage unit configured to store a plurality of prediction models used to predict an upcoming stage of carotid stenosis; and a predicting unit configured to obtain an outcome by applying a value indicating a stage of carotid stenosis corresponding to the result of carotid stenosis ultrasonography and the extracted risk score factor to a prediction model corresponding to the determined cluster to which the patient's medical test data belong among the plurality of prediction models.

The extracted risk factor score may include either one or both of a blood pressure level and a cholesterol level.

In another general aspect, a method of predicting an upcoming stage carotid stenosis includes receiving a patient's medical test data relating to carotid stenosis; determining a cluster to which the patient's medical test data belong based on a gender of the patient; extracting from the patient's medical test data a risk factor score including a result of carotid stenosis ultrasonography; storing a plurality of prediction models used to predict an upcoming stage of carotid stenosis; and obtaining an outcome by applying a value indicating a stage of carotid stenosis corresponding to the result of carotid stenosis ultrasonography and the extracted risk factor score to the prediction model corresponding to the determined cluster to which the patient's medical test data belong among the plurality of prediction models.

The extracted risk factor score may include either one or both of a blood pressure level and a cholesterol level.

In another general aspect, an apparatus for predicting an upcoming stage of carotid stenosis includes a receiving unit configured to receive a patient's medical test data relating to carotid stenosis and corresponding operation information; a cluster determining unit configured to determine a cluster to which the patient's medical test data belong based on a characteristic of the patient; a risk factor score extracting unit configured to extract from the patient's medical test data at least one risk factor score of a risk factor of a risk factor set of the determined cluster to which the patient's medical test data belong; a prediction model storage unit configured to store a plurality of prediction models used to predict an upcoming stage of carotid stenosis; a prediction model learning unit configured to perform machine learning by applying the extracted risk factor score to a prediction model corresponding to the determined cluster to which the patient's medical test data belong among the plurality of prediction models; and a predicting unit configured to obtain an outcome by applying the extracted risk factor score to the prediction model corresponding to the determined cluster to which the patient's medical test data belong.

The prediction model learning unit may be further configured to perform the machine learning when the operation information is a learning instruction; and the predicting unit may be further configured to obtain the outcome when the operation information is a predicting instruction.

The extracted risk factor score may include a result of carotid stenosis ultrasonography and a corresponding test date.

The prediction model learning unit may be further configured to classify all results of carotid stenosis ultrasonography into at least two sections; and each section of the at least two sections may be representative of a specific stage of carotid stenosis.

The prediction model learning unit may be further configured to assign a first outcome to the patient's medical test data when a stage of carotid stenosis corresponding to a last result of carotid stenosis ultrasonography of the patient's medical test data is higher than a stage of carotid stenosis corresponding to a first result of carotid stenosis ultrasonography of the patient's medical test data; and assign a second outcome to the patient's medical test data in other cases.

When the predicting unit obtains the first outcome when the patient's medical test data is received with the predicting instruction, a stage of carotid stenosis of the patient may be predicted to be heightened; and when the predicting unit obtains the second outcome when the patient's medical test data is received with the predicting instruction, the stage of carotid stenosis of the patient may be predicted not to be heightened.

The extracted risk factor score may include at least two results of carotid stenosis ultrasonography; and the prediction model learning unit may be further configured to perform the machine learning using the at least two results of carotid stenosis ultrasonography.

In another general aspect, a method of predicting an upcoming stage of carotid stenosis includes receiving a patient's medical test data relating to carotid stenosis and corresponding operation information; determining a cluster to which the patient's medical test data belong based on a characteristic of the patient; extracting from the patient's medical test data at least one risk factor score of a risk factor of a risk factor set of the determined cluster to which the patient's medical test data belong; and selectively performing machine learning or performing prediction using a prediction model according to the operation information.

The selectively performing of the machine learning or performing the prediction may include, when the operation information is a learning instruction, performing the machine learning by applying the extracted risk factor score to a prediction model corresponding to the determined cluster to which the patient's medical test data belong among a plurality of prediction models used for predicting an upcoming stage of carotid stenosis; and, when the operation information is a predicting instruction, performing the prediction using a prediction model by applying the extracted risk factor score to the prediction model corresponding to the determined cluster to which the patient's medical test data belong.

The extracted risk factor score may include a result of carotid stenosis ultrasonography and a corresponding test date.

The performing of the machine learning may include classifying all results of carotid stenosis ultrasonography into at least two sections; and each section of the at least two sections may be representative of a specific stage of carotid stenosis.

The performing of the machine learning may further include assigning a first outcome to the patient's medical test data when a stage of carotid stenosis corresponding to a last result of carotid stenosis ultrasonography of the patient's medical test data is higher than a stage of carotid stenosis corresponding to a first result of carotid stenosis ultrasonography of the patient's medical test data; and assigning a second outcome to the patient's medical test data in other cases.

When the performing of the prediction using a prediction model obtains the first outcome when the patient's medical test data is received with the predicting instruction, a stage of carotid stenosis of the patient may be predicted to be heightened; and when the performing of the prediction using a prediction model obtains the second outcome when the patient's medical test data is received with the predicting instruction, the stage of carotid stenosis of the patient may be predicted not to be heightened.

Other features and aspects will be apparent from the following detailed description, the drawings, and the claims.

### SUMMARY

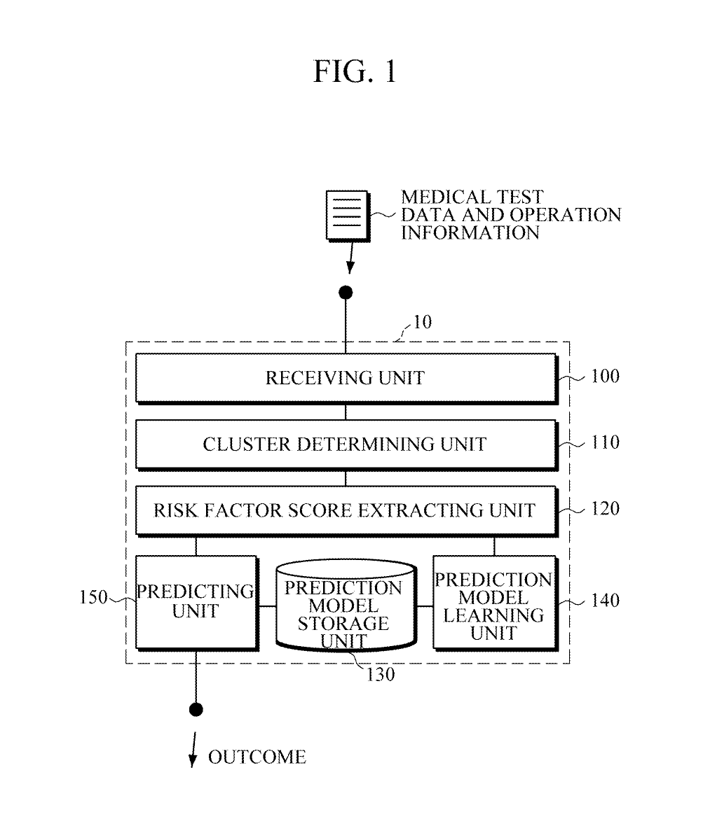

FIG. 1 is a block diagram illustrating an example of an apparatus for predicting an upcoming stage of carotid stenosis.

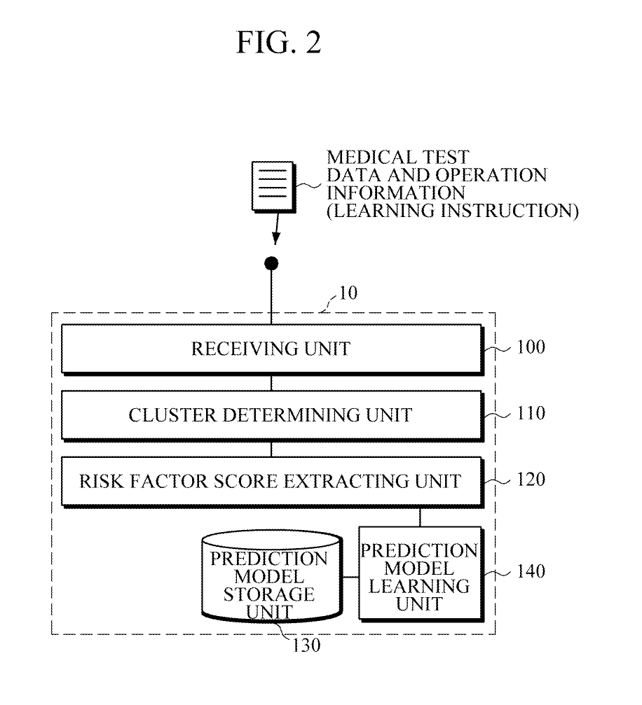

FIG. 2 is a block diagram illustrating an example of elements of the apparatus for predicting an upcoming stage of carotid stenosis shown in FIG. 1 that are activated when operation information is a learning instruction.

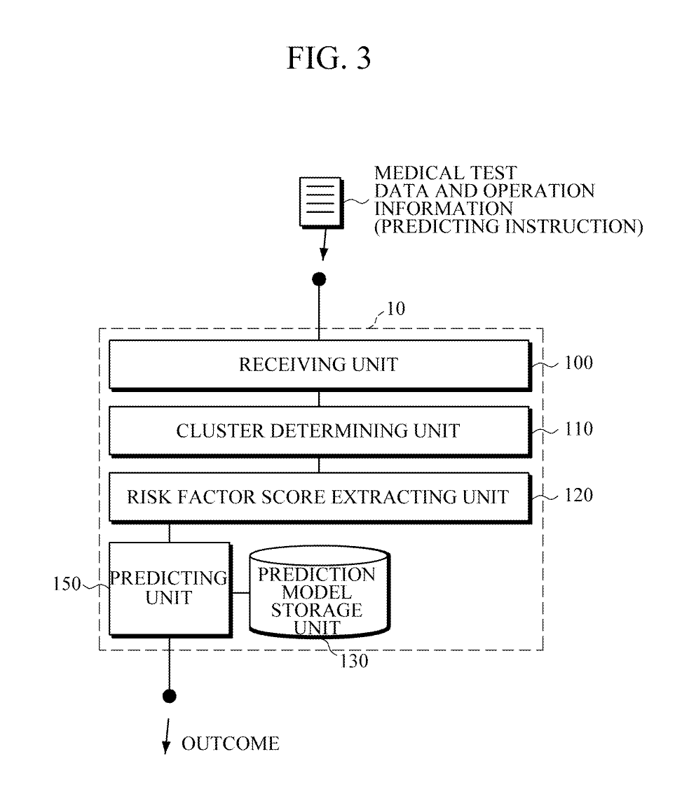

FIG. 3 is a block diagram illustrating an example of elements of the apparatus for predicting an upcoming stage of carotid stenosis shown in FIG. 1 that are activated when operation information is a predicting instruction.

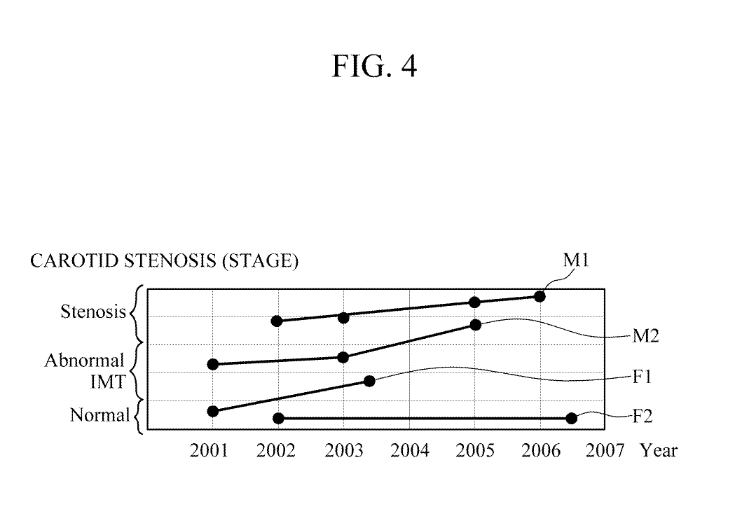

FIG. 4 is a graph for explaining an example of a carotid stenosis progression in phases.

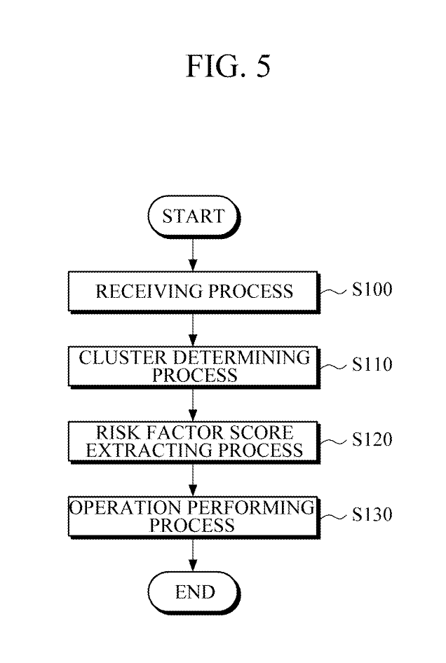

FIG. 5 is a flow chart illustrating an example of a method of predicting an upcoming stage of carotid stenosis.

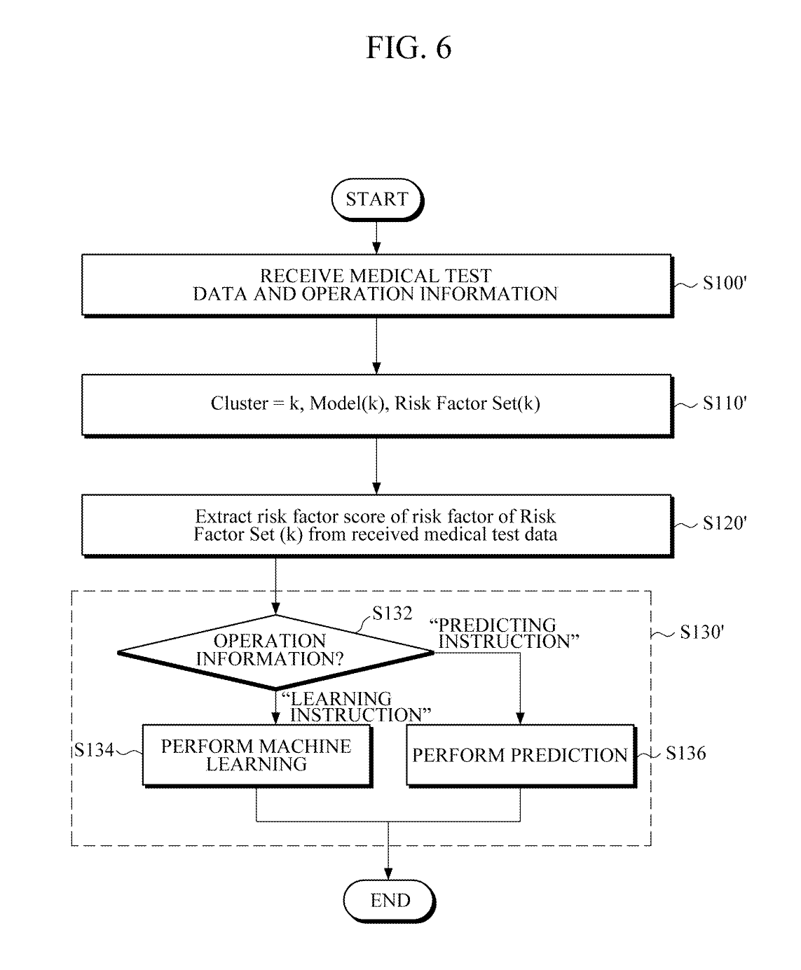

FIG. 6 is a flow chart illustrating a detailed example of the method of FIG. 5.

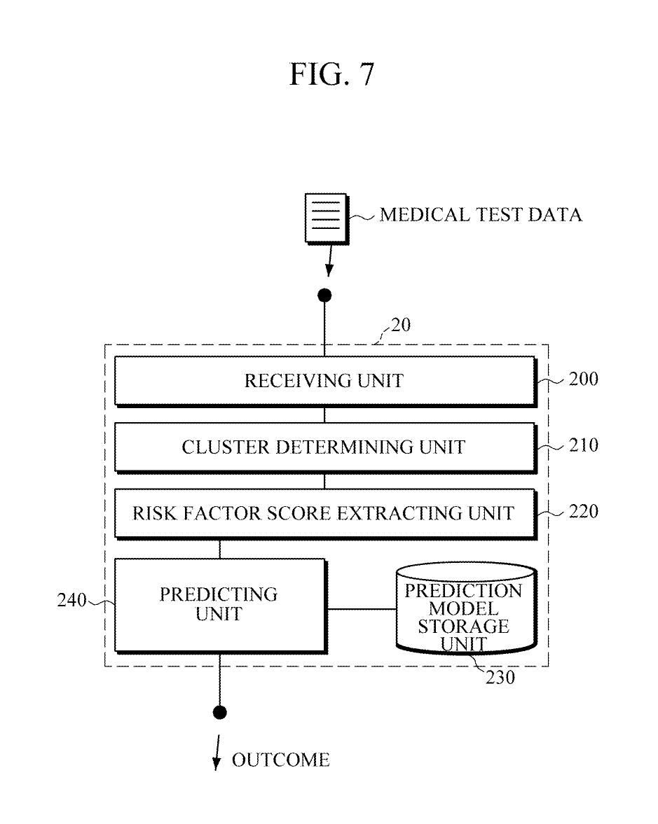

FIG. 7 is a block diagram illustrating another example of an apparatus for predicting an upcoming stage of carotid stenosis.

### DETAILED DESCRIPTION

The following description is provided to assist the reader in gaining a comprehensive understanding of the methods, apparatuses, and/or systems described herein. However, various changes, modifications, and equivalents of the methods, apparatuses, and/or systems described herein will be apparent to one of ordinary skill in the art. Also, descriptions of functions and constructions that are well known to one of ordinary skill in the art may be omitted for increased clarity and conciseness.

Throughout the drawings and the detailed description, the same reference numerals refer to the same elements. The drawings may not be to scale, and the relative size, proportions, and depiction of elements in the drawings may be exaggerated for clarity, illustration, and convenience.

The following two conditions enable a patient's upcoming stage of carotid stenosis to be predicted.

First, a prediction model (a prediction model used for predicting an upcoming stage of carotid stenosis) needs to be learned for predicting a patient's upcoming stage of carotid stenosis based on data from various medical tests, including an ultrasound diagnosis.

Second, an outcome indicative of an upcoming stage of carotid stenosis needs to be obtained by applying a specific patient's medical test data to the learned prediction model.

FIG. 1 is a block diagram illustrating an example of an apparatus for predicting an upcoming stage of carotid stenosis.

Referring to FIG. 1, the apparatus 10 for predicting an upcoming stage of carotid stenosis includes a receiving unit 100, a cluster determining unit 110, a risk factor score extracting unit 120, a prediction model storage unit 130, a prediction model learning unit 140, and a predicting unit 150.

The receiving unit 100 receives a patient's medical test data relating to carotid stenosis, and corresponding operation information.

Medical test data is a collection of data about various medical tests and diagnoses with respect to a patient. A risk factor included in the medical test data may or may not be directly related to carotid stenosis progression. Therefore, only a value of a risk factor (that is, a risk factor score) that has a profound statistical significance for carotid stenosis progression should be selectively extracted and then applied to a prediction model for predicting an upcoming stage of carotid stenosis.

Operation information is an instruction that points out a type of an operation to be performed with respect to received medical test data. For example, the operation information may be an instruction that is input by selecting an appropriate operation button in a menu of the apparatus for predicting an upcoming stage of carotid stenosis.

If the operation information is a learning instruction, the prediction model learning unit 140 performs machine learning using the medical test data. Alternatively, if the operation information is a predicting instruction, the predicting unit 150 predicts an upcoming stage of carotid stenosis using the medical test data.

The cluster designating unit 110 determines a cluster to which the patient's medical test data belong based on at least one characteristic of the patient. The at least one characteristic of the patient may be included in the patient's medical test data.

A cluster is a collection of medical test data having a common characteristic. Thus, if a prediction model optimized for all of the medical test data belonging to the same cluster is used to perform a prediction based on new medical test data belonging to that cluster, prediction accuracy may improve profoundly.

For example, patients may be classified into two clusters according to gender. Thus, any received medical test data belong to either a male cluster or to a female cluster.

The risk factor score extracting unit 120 extracts from the medical test data at least one risk factor score of a risk factor of a risk factor set of the determined cluster to which the medical test data belong.

A risk factor is a factor that affects an upcoming stage of carotid stenosis. For example, a first result of carotid stenosis ultrasonography, a blood pressure level, and cholesterol level are highly significant risk factors. A value of a risk factor, that is, a risk factor score, is included in the medical test data. For example, if a risk factor is age, a patient's age (for example, 30) is included in the medical test data as a risk factor score. A different risk factor set may be applied to each prediction model.

Table 1 below shows a format of a risk factor set applied to a prediction model.

TABLE 1

| Risk Factor Set | Model | Risk Factor | OR | CI (95%) | | P-Value |
|---|---|---|---|---|---|---|
| RFS₁ | Model₁ | RF₁ | 1.01 | 1.01 | 1.01 | 3.54E−07 |
| | | RF₂ | 0.13 | 0.11 | 0.15 | <2E−16 |
| | | . . . | . . . | . . . | . . . | . . . |
| . . . | . . . | . . . | . . . | . . . | . . . | . . . |

A risk factor set RFS, shown in Table 2 consists of a plurality of risk factors RF₁, RF_{2, . . . ,} and the like. Among other factors configuring each medical test data, a factor closely related to progression of carotid stenosis is utilized as a risk factor. For example, a risk factor set may include a value of a significant risk factor, such as the first result of carotid stenosis ultrasonography, a blood pressure level, an age, a value determined according to whether the patient is a smoker or a non-smoker, a value determined according to whether the patient has diabetes, a LDL cholesterol level, and an HDL cholesterol level.

Each risk factor may include additional fields to show an odds ratio (OR) between the risk factor and a carotid stenosis progression, a confidence interval of the OR (for example, a confidence level of 95%) and a statistical significance (P-Value) of the OR.

The prediction model storage unit 130 stores a plurality of prediction models used for predicting an upcoming stage of carotid stenosis.

A different prediction model is employed with respect to each cluster. Therefore, if there are a plurality of clusters, a plurality of corresponding prediction models are provided. The plurality of prediction models are stored in the prediction model storage unit 130. In addition, if an operation is performed with respect to medical test data belonging to a specific cluster, a prediction model corresponding to the specific cluster is selected to be used.

The prediction model learning unit 140 performs machine learning by applying the risk factor score extracted by the risk factor score extracting unit 120 to the prediction model corresponding to the cluster to which the medical test data belong.

A wide range of machine learning algorithms may be used to perform machine learning on each prediction model. For example, a support vector machine (SVC), a decision tree, a multilayer perceptron (MLP), a LogitBoost, or any other machine learning algorithm known to one of ordinary skill in the art, or any combination thereof may be used.

A prediction model may be learned with respect to a patient's medical test data by receiving the medical test data and then performing machine learning on the prediction model using a specific algorithm. Next, if a patient's new medical test data is received, the prediction model may be employed to predict the patient's progression of carotid stenosis based on the new medical test data.

The predicting unit 150 obtains an outcome indicative of the patient's upcoming stage of carotid stenosis by applying a risk factor score extracted from the medical test data by the risk factor extracting unit 120 to a prediction model corresponding to the cluster to which the medical test data belong.

When medical test data is received in the apparatus 10 for predicting an upcoming stage of carotid stenosis shown in FIG. 1, the medical test data may be used to perform machine learning on a corresponding prediction model or to predict an upcoming stage of carotid stenosis. FIGS. 2 and 3 illustrate each of these cases.

FIG. 2 is a diagram illustrating an example of elements of the apparatus 10 for predicting an upcoming stage of carotid stenosis shown in FIG. 1 that are activated when operation information is a learning instruction.

If operation information is a learning instruction, elements of the apparatus 10 for predicting an upcoming stage of carotid stenosis that are used in performing machine learning on a prediction model using received medical test data are activated. These elements include the receiving unit 100, the cluster determining unit 110, the risk factor score extracting unit 120, the prediction model storage unit 130, and the prediction model learning unit 140 as shown in FIG. 2.

FIG. 3 is a diagram illustrating an example of elements of the apparatus 10 for predicting an upcoming stage of carotid stenosis shown in FIG. 1 that are activated when operation information is a predicting instruction.

If operation information is a predicting instruction, elements of the apparatus 10 for predicting an upcoming stage of carotid stenosis that are used in predicting an upcoming stage of carotid stenosis by applying received medical test data to a prediction model corresponding to a cluster to which the medical test data belong are activated. These elements include the receiving unit 100, the cluster determining unit 110, the risk factor score extracting unit 120, the prediction model storage unit 130, and the prediction model learning unit 140.

FIG. 4 is a graph for explaining a carotid stenosis progression in phases.

A progression or a stage of carotid stenosis may be represented by a value or a section. A diagnosis environment or a patient's condition and other characteristics may cause deviation among patient's medical test data, so dividing carotid stenosis progression into sections may be more accurate.

In FIG. 4, patients are classified into a female cluster and a male cluster according to gender. A male patient M1 was tested in 2002, 2003, 2005, and 2006 for a stage of carotid stenosis. A male patient M2 was tested in 2001, 2003, and 2005, a female patient F1 was tested in 2001 and 2003, and a female patient F2 was tested in 2002 and 2006.

According to results of carotid stenosis ultrasonography, a progression of carotid progression may be classified into a normal state, an abnormal intima-media thickness (abnormal IMT) state, and a stenosis state.

The further carotid stenosis has progressed, the higher a stage is assigned to the state of carotid stenosis. For example, stage 1 of carotid stenosis corresponds to the normal state, stage 2 of carotid stenosis corresponds to the abnormal IMT state, and stage 3 of carotid stenosis corresponds to the stenosis state.

A result of carotid stenosis ultrasonography and a corresponding test date are risk factors that may be used for performing machine learning on a prediction model and employing the learned prediction model for predicting an upcoming stage of carotid stenosis.

Specifically, a prediction model should be learned first. If at least two risk factor scores, each including a result of carotid stenosis ultrasonography and a corresponding test date, are extracted from medical test data, learning may be performed on a prediction model by applying the first result of carotid stenosis ultrasonography and the last result of carotid stenosis ultrasonography, along with other risk factor scores included in the medical test data, to the prediction model corresponding to a cluster to which the medical test data belong.

In addition, if a specific result of carotid stenosis ultrasonography and a corresponding test date are extracted from medical test data, an outcome indicative of a carotid stenosis progression at a specific point in time may be obtained by applying the specific result of carotid stenosis ultrasonography and the corresponding test date, along with other risk factor scores included in the medical test data, to the prediction model corresponding to the cluster to which the medical test data belong. The specific point in time may be, for example, four years in the future. However, four years is merely one example, and other time periods may be used.

Table 2 below shows stages of carotid stenosis that may be found by comparing the first result of carotid stenosis ultrasonography and the last result of carotid stenosis ultrasonography for the patients shown in FIG. 4.

TABLE 2

| | Stage of Carotid | Stage of Carotid | |
|---|---|---|---|
| | Stenosis at First | Stenosis at Last | |
| Patient | Ultrasonography | Ultrasonography | Change in Stage |
| M1 | Stage 3 | Stage 3 | No Change (±0) |
| M2 | Stage 2 | Stage 3 | Heightened (±1) |
| F1 | Stage 1 | Stage 2 | Heightened (±1) |
| F2 | Stage 1 | Stage 1 | No Change (±0) |

Referring again to FIG. 1, the prediction model learning unit 140 determines whether a stage of carotid stenosis corresponding to the last result of carotid stenosis ultrasonography included in medical test data is higher than a stage of carotid stenosis corresponding to the first result of carotid stenosis ultrasonography included in medical test data. For example, a stage of carotid stenosis has been heightened with respect to a male patient M2 and a female patient F1 in Table 2. In contrast, there has been no change in a state of carotid stenosis with respect to a male patient M1 and a female patient F1 in Table 2.

The fact that a stage of carotid stenosis has been heightened for patients M2 and F1 indicates that carotid stenosis has progressed further during the period between the first carotid stenosis ultrasonography and the last carotid stenosis ultrasonography. In this case, the prediction model learning unit 140 may assign “1”, for example, an outcome indicating information about an upcoming stage of carotid stenosis with respect to the medical test data.

Alternatively, the fact that a stage of carotid stenosis has not been heightened for patients M1 and F2 indicates that a stage of carotid stenosis has been maintained at a relatively constant level or that carotid stenosis has been reduced or eliminated. In this case, the prediction model learning unit 140 may assign “0”, for example, as an outcome indicating information about an upcoming stage of carotid stenosis.

As such, if machine learning is performed on a prediction model by the prediction model learning unit 140 using medical test data, the prediction model may be employed to predict an upcoming stage of carotid stenosis for different medical test data belonging to the same cluster as the medical test data used to perform the machine learning.

That is, if the predicting unit 150 obtains an outcome of “1” when a patient's medical test data is received with a predicting instruction, the patient's stage of carotid stenosis is predicted to be heightened. For example, if a result of carotid stenosis ultrasonography included in a patient's medical test data corresponds to the above-mentioned “abnormal IMT” state, a stage of carotid stenosis is predicted to be heightened to become the above-mentioned “stenosis” state, meaning that carotid stenosis is predicted to progress further.

In contrast, if the predicting unit 150 obtains an outcome of “0” when a patient's medical test data is received with a predicting instruction, the patient's stage of carotid stenosis is predicted not to increase. For example, if a result of carotid stenosis ultrasonography included in a patient's medical test data corresponds to the “abnormal IMT” state, a stage of carotid stenosis is predicted to remain at the “abnormal IMT” state.

Machine learning is performed on a prediction model only when medical test data is a result of two or more carotid stenosis ultrasonographies. If a carotid stenosis ultrasonography has not been performed, or has been performed only once, it is impossible to extract a first result of carotid stenosis ultrasonography and a last result of carotid stenosis ultrasonography from the medical test data because a carotid stenosis ultrasonography either has not been performed at all, or has been performed only once.

In the above example, patients are classified into a male cluster and a female cluster, but this is merely one example. In other words, patients' medical test data may be clustered in various ways with a variety of existing clustering techniques.

However, there may be numerous standards reflecting a common characteristic between all of the medical test data belonging to the same cluster, and how to apply such standards (or a combination of the standards) may determine a type of a clustering technique to be used.

For example, patients' medical test data may be classified into k clusters using a k-means clustering algorithm. Other clustering techniques or algorithms may be used to cluster patients' medical test data properly.

FIG. 5 is a flow chart illustrating an example of a method of predicting an upcoming stage of carotid stenosis. Referring to FIG. 5, the method of predicting an upcoming stage of carotid stenosis includes a receiving process S100, a cluster determining process S110, a risk factor score extracting process S120, and an operation performing process S130.

In the receiving process S100, a patient's medical test data related to carotid stenosis and corresponding operation information are received in an apparatus for predicting an upcoming stage of carotid stenosis.

In the cluster determining process S110, clustering is performed on the medical test data received in the receiving process S100 based on at least one characteristic of the patient. The at least one characteristic of the patient may be included in the medical test data. Accordingly, a cluster to which the medical test data belong is determined.

For example, if medical test data are classified into two clusters based on gender as a characteristic of the patient, received medical test data of a male patient belongs to a male cluster, and received medical test data of a female patient belongs to a female cluster.

In the risk factor score extracting process S120, at least one risk factor score of a risk factor of a risk factor set of the cluster to which the medical test data belong is extracted from the medical test data.

In the operation performing process S130, machine learning or prediction using a prediction model is selectively performed according to the operation information that was received in the receiving process S100.

FIG. 6 is a flow chart illustrating a detailed example of the method of FIG. 5. Processes S100′, S110′, S120′, and S130′ of FIG. 6 respectively correspond to processes S100, S110, S120, and S130 of FIG. 5.

In a receiving process S100′, a patient's medical test data and corresponding operation information are received in a receiving unit of an apparatus for predicting an upcoming stage of carotid stenosis.

In a cluster determining process S110′, a cluster to which the medical test data belong is determined based on at least one characteristic of the patient. The at least one characteristic of the patient may be included in the medical test data. In addition, a prediction model to be used with respect to the medical test data is determined.

For example, when a clustering technique requiring N clusters (1<k≦N) is used and the received medical test data belong to a k-th cluster, a prediction model Model (k) and a risk factor set Risk Factor Set (k) each corresponding to the k-th cluster are used. The Risk Factor Set (k) is a set of risk factors of the medical test data of the k-th cluster.

In a risk factor score extracting process S120′, a risk factor score of a risk factor of the Risk Factor Set (k) is extracted from the received medical test data. Different prediction model and different risk factor sets are used with respect to medical test data belonging to different clusters. Conversely, the same prediction model and the same risk factor set are used with respect to medical test data belonging to the same cluster.

An operation performing process S130′ includes an operation information determining process S132.

When the operation information is a “learning instruction”, the risk factor score extracted in the process S120′ for extracting a risk factor score is applied to the prediction model corresponding to a cluster to which the received medical test data belong to perform machine learning on the prediction model in a machine learning process S134.

When the operation information is a “predicting instruction”, the risk factor score extracted in the risk factor score extracting process S120′ is applied to a prediction model corresponding to a cluster to which the received medical test data belong to predict a patient's upcoming stage of carotid stenosis in a predicting process S136. The upcoming stage of carotid stenosis may be predicted at a specific point in time, for example, four years in the future. However, four years is merely one example, and other time periods may be used.

In the operation performing process S130′, results of carotid stenosis ultrasonography are classified into two or more sections, and each section is representative of a specific stage of carotid stenosis. In this way, it is possible to determine which stage of carotid stenosis a result of carotid stenosis ultrasonography extracted from medical test data corresponds to. That is, a carotid stenosis progression may be presented in phases.

If carotid stenosis progression is presented in phases, a stage of carotid stenosis corresponding to a last result of carotid stenosis ultrasonography extracted from medical test data is compared with a stage of carotid stenosis corresponding to a first result of carotid stenosis ultrasonography extracted from the medical test data. If the stage of carotid stenosis corresponding to the last result of carotid stenosis ultrasonography is higher than the stage of carotid stenosis corresponding to the first result of carotid stenosis ultrasonography, an outcome (for example, “b 1”) indicating that a stage of carotid stenosis is predicted to be heightened is assigned. An outcome (for example, “0” or a value other than “1”) indicating that a stage of carotid stenosis is not predicted to be heightened is assigned.

If machine learning is performed on a prediction model with respect to medical test data in the machine learning process S134 to present carotid stenosis progression in phases, the prediction model may be used to predict an upcoming stage of carotid stenosis based on different medical test data.

For example, if an outcome of “1” is obtained with respect to different medical test data in the predicting process S136, it may be predicted that a stage of carotid stenosis will be heightened. Alternatively, if an outcome of “0” is obtained with respect to the different medical test data, it may be predicted that a stage of carotid stenosis will not be heightened.

FIG. 7 is a block diagram illustrating another example of an apparatus for predicting an upcoming stage of carotid stenosis. In the example of FIG. 7, the apparatus 20 for predicting an upcoming stage of carotid stenosis does not perform machine learning on a plurality of prediction models stored in a prediction model storage unit.

The apparatus 20 for predicting an upcoming stage of carotid stenosis includes a receiving unit 200, a cluster determining unit 210, a risk factor score extracting unit 220, a prediction model storage unit 230, and a predicting unit 240.

The receiving unit 200 receives a patient's medical test data relating to carotid stenosis. Since machine learning on a prediction model is not performed in the apparatus 20 for predicting an upcoming stage of carotid stenosis, it is unnecessary to receive operation information.

The cluster determining unit 210 determines a cluster to which the received medical test data belong based on a gender of the patient. The gender may be included in the medical test data.

The risk factor score extracting unit 220 extracts from the medical test data at least one risk factor score including a result of carotid stenosis ultrasonography. In addition, the at least one risk factor score may include either one or both of a blood pressure level and a cholesterol level.

The prediction model storage unit 230 stores a plurality of prediction models used for predicting an upcoming stage of carotid stenosis.

The predicting unit 240 obtains an outcome by applying a value indicating a stage of carotid stenosis corresponding to a result of carotid stenosis ultrasonography to a prediction model corresponding to the determined cluster to which the medical test data belong.

If an outcome of “1” is obtained, this indicates that a stage of carotid stenosis is predicted to be heightened, indicating that the patient's upcoming stage of carotid stenosis is predicted to increase. Conversely, if an outcome of “0” is obtained, this indicates that a stage of carotid stenosis is predicted to not be heightened, indicating that the patient's upcoming of carotid stenosis is predicted to not increase.

As described above, if it is possible to predict whether a patient's carotid stenosis progression will become worse at a specific point in time (for example, four years in the future), high-risk patients may be appropriately treated with medication so that heart diseases, strokes, and other cardiovascular problems may be effectively prevented. In addition, the prediction may help low-risk patients avoid unnecessary medical tests and excessive preventive treatment.

The receiving unit 100, the cluster determining unit 110, the risk factor score extracting unit 120, the prediction model storage unit 130, the prediction model learning unit 140, the predicting unit 150, the receiving unit 200, the cluster determining unit 210, the risk factor score extracting unit 220, the prediction model storage unit 230, and the predicting unit 240 described above that perform the operations illustrated in FIGS. 5 and 6 may be implemented using one or more hardware components, one or more software components, or a combination of one or more hardware components and one or more software components.

A hardware component may be, for example, a physical device that physically performs one or more operations, but is not limited thereto. Examples of hardware components include resistors, capacitors, inductors, power supplies, frequency generators, operational amplifiers, power amplifiers, low-pass filters, high-pass filters, band-pass filters, analog-to-digital converters, digital-to-analog converters, and processing devices.

A software component may be implemented, for example, by a processing device controlled by software or instructions to perform one or more operations, but is not limited thereto. A computer, controller, or other control device may cause the processing device to run the software or execute the instructions. One software component may be implemented by one processing device, or two or more software components may be implemented by one processing device, or one software component may be implemented by two or more processing devices, or two or more software components may be implemented by two or more processing devices.

A processing device may be implemented using one or more general-purpose or special-purpose computers, such as, for example, a processor, a controller and an arithmetic logic unit, a digital signal processor, a microcomputer, a field-programmable array, a programmable logic unit, a microprocessor, or any other device capable of running software or executing instructions. The processing device may run an operating system (OS), and may run one or more software applications that operate under the OS. The processing device may access, store, manipulate, process, and create data when running the software or executing the instructions. For simplicity, the singular term “processing device” may be used in the description, but one of ordinary skill in the art will appreciate that a processing device may include multiple processing elements and multiple types of processing elements. For example, a processing device may include one or more processors, or one or more processors and one or more controllers. In addition, different processing configurations are possible, such as parallel processors or multi-core processors.

A processing device configured to implement a software component to perform an operation A may include a processor programmed to run software or execute instructions to control the processor to perform operation A. In addition, a processing device configured to implement a software component to perform an operation A, an operation B, and an operation C may have various configurations, such as, for example, a processor configured to implement a software component to perform operations A, B, and C; a first processor configured to implement a software component to perform operation A, and a second processor configured to implement a software component to perform operations B and C; a first processor configured to implement a software component to perform operations A and B, and a second processor configured to implement a software component to perform operation C; a first processor configured to implement a software component to perform operation A, a second processor configured to implement a software component to perform operation B, and a third processor configured to implement a software component to perform operation C; a first processor configured to implement a software component to perform operations A, B, and C, and a second processor configured to implement a software component to perform operations A, B, and C, or any other configuration of one or more processors each implementing one or more of operations A, B, and C. Although these examples refer to three operations A, B, C, the number of operations that may implemented is not limited to three, but may be any number of operations required to achieve a desired result or perform a desired task.

Software or instructions for controlling a processing device to implement a software component may include a computer program, a piece of code, an instruction, or some combination thereof, for independently or collectively instructing or configuring the processing device to perform one or more desired operations. The software or instructions may include machine code that may be directly executed by the processing device, such as machine code produced by a compiler, and/or higher-level code that may be executed by the processing device using an interpreter. The software or instructions and any associated data, data files, and data structures may be embodied permanently or temporarily in any type of machine, component, physical or virtual equipment, computer storage medium or device, or a propagated signal wave capable of providing instructions or data to or being interpreted by the processing device. The software or instructions and any associated data, data files, and data structures also may be distributed over network-coupled computer systems so that the software or instructions and any associated data, data files, and data structures are stored and executed in a distributed fashion.

For example, the software or instructions and any associated data, data files, and data structures may be recorded, stored, or fixed in one or more non-transitory computer-readable storage media. A non-transitory computer-readable storage medium may be any data storage device that is capable of storing the software or instructions and any associated data, data files, and data structures so that they can be read by a computer system or processing device. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, or any other non-transitory computer-readable storage medium known to one of ordinary skill in the art.

Functional programs, codes, and code segments for implementing the examples disclosed herein can be easily constructed by a programmer skilled in the art to which the examples pertain based on the drawings and their corresponding descriptions as provided herein.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. An apparatus for predicting an upcoming stage of carotid stenosis, the apparatus comprising:
a receiving unit configured to receive a patient's medical test data relating to carotid stenosis;
a cluster determining unit configured to determine a cluster to which the patient's medical test data belong based on a gender of the patient;
a risk factor score extracting unit configured to extract from the patient's medical test data a risk factor score comprising a result of carotid stenosis ultrasonography;
a prediction model storage unit configured to store a plurality of prediction models used to predict an upcoming stage of carotid stenosis; and
a predicting unit configured to obtain an outcome by applying a value indicating a stage of carotid stenosis corresponding to the result of carotid stenosis ultrasonography and the extracted risk score factor to a prediction model corresponding to the determined cluster to which the patient's medical test data belong among the plurality of prediction models.

2. The apparatus of claim 1, wherein the extracted risk factor score comprises either one or both of a blood pressure level and a cholesterol level.

3. A method of predicting an upcoming stage carotid stenosis, the method comprising:
receiving a patient's medical test data relating to carotid stenosis;
determining a cluster to which the patient's medical test data belong based on a gender of the patient;
extracting from the patient's medical test data a risk factor score comprising a result of carotid stenosis ultrasonography;
storing a plurality of prediction models used to predict an upcoming stage of carotid stenosis; and
obtaining an outcome by applying a value indicating a stage of carotid stenosis corresponding to the result of carotid stenosis ultrasonography and the extracted risk factor score to the prediction model corresponding to the determined cluster to which the patient's medical test data belong among the plurality of prediction models.

4. The method of claim 3, wherein the extracted risk factor score comprises either one or both of a blood pressure level and a cholesterol level.

5. An apparatus for predicting an upcoming stage of carotid stenosis, the apparatus comprising:
a receiving unit configured to receive a patient's medical test data relating to carotid stenosis and corresponding operation information;
a cluster determining unit configured to determine a cluster to which the patient's medical test data belong based on a characteristic of the patient;
a risk factor score extracting unit configured to extract from the patient's medical test data at least one risk factor score of a risk factor of a risk factor set of the determined cluster to which the patient's medical test data belong;
a prediction model storage unit configured to store a plurality of prediction models used to predict an upcoming stage of carotid stenosis;
a prediction model learning unit configured to perform machine learning by applying the extracted risk factor score to a prediction model corresponding to the determined cluster to which the patient's medical test data belong among the plurality of prediction models; and
a predicting unit configured to obtain an outcome by applying the extracted risk factor score to the prediction model corresponding to the determined cluster to which the patient's medical test data belong.

6. The apparatus of claim 5, wherein the prediction model learning unit is further configured to perform the machine learning when the operation information is a learning instruction; and
the predicting unit is further configured to obtain the outcome when the operation information is a predicting instruction.

7. The apparatus of claim 6, wherein the extracted risk factor score comprises a result of carotid stenosis ultrasonography and a corresponding test date.

8. The apparatus of claim 7, wherein the prediction model learning unit is further configured to classify all results of carotid stenosis ultrasonography into at least two sections; and
each section of the at least two sections is representative of a specific stage of carotid stenosis.

9. The apparatus of claim 8, wherein the prediction model learning unit is further configured to:
assign a first outcome to the patient's medical test data when a stage of carotid stenosis corresponding to a last result of carotid stenosis ultrasonography of the patient's medical test data is higher than a stage of carotid stenosis corresponding to a first result of carotid stenosis ultrasonography of the patient's medical test data; and
assign a second outcome to the patient's medical test data in other cases.

10. The apparatus of claim 9, wherein when the predicting unit obtains the first outcome when the patient's medical test data is received with the predicting instruction, a stage of carotid stenosis of the patient is predicted to be heightened; and
when the predicting unit obtains the second outcome when the patient's medical test data is received with the predicting instruction, the stage of carotid stenosis of the patient is predicted not to be heightened.

11. The apparatus of claim 10, wherein the extracted risk factor score comprises at least two results of carotid stenosis ultrasonography; and
the prediction model learning unit is further configured to perform the machine learning using the at least two results of carotid stenosis ultrasonography.

12. A method of predicting an upcoming stage of carotid stenosis, the method comprising:
receiving a patient's medical test data relating to carotid stenosis and corresponding operation information;
determining a cluster to which the patient's medical test data belong based on a characteristic of the patient;
extracting from the patient's medical test data at least one risk factor score of a risk factor of a risk factor set of the determined cluster to which the patient's medical test data belong; and
selectively performing machine learning or performing prediction using a prediction model according to the operation information.

13. The method of claim 12, wherein the selectively performing of the machine learning or performing the prediction comprises:
when the operation information is a learning instruction, performing the machine learning by applying the extracted risk factor score to a prediction model corresponding to the determined cluster to which the patient's medical test data belong among a plurality of prediction models used for predicting an upcoming stage of carotid stenosis; and
when the operation information is a predicting instruction, performing the prediction using a prediction model by applying the extracted risk factor score to the prediction model corresponding to the determined cluster to which the patient's medical test data belong.

14. The method of claim 13, wherein the extracted risk factor score comprises a result of carotid stenosis ultrasonography and a corresponding test date.

15. The method of claim 14, wherein the performing of the machine learning comprises classifying all results of carotid stenosis ultrasonography into at least two sections; and
each section of the at least two sections is representative of a specific stage of carotid stenosis.

16. The method of claim 15, wherein the performing of the machine learning further comprises:
assigning a first outcome to the patient's medical test data when a stage of carotid stenosis corresponding to a last result of carotid stenosis ultrasonography of the patient's medical test data is higher than a stage of carotid stenosis corresponding to a first result of carotid stenosis ultrasonography of the patient's medical test data; and
assigning a second outcome to the patient's medical test data in other cases.

17. The method of claim 16, wherein when the performing of the prediction using a prediction model obtains the first outcome when the patient's medical test data is received with the predicting instruction, a stage of carotid stenosis of the patient is predicted to be heightened; and
when the performing of the prediction using a prediction model obtains the second outcome when the patient's medical test data is received with the predicting instruction, the stage of carotid stenosis of the patient is predicted not to be heightened.
